(19) United States
(12) Patent Application Publication
(10) Pub. No.: US 2023/0277798 A1
(43) Pub. Date: Sep. 7, 2023

(54) DEVICE AND METHOD FOR REGULATING A GAS FLOW

(71) Applicant: Loewenstein Medical Technology S.A., Luxembourg (LU)

(72) Inventors: Benjamin Adametz, Hamburg (DE); Ralf Froehlich, Schenefeld (DE)

(21) Appl. No.: 18/175,857

(22) Filed: Feb. 28, 2023

(30) Foreign Application Priority Data
Mar. 2, 2022 (DE) 102022104938.8

(51) Int. Cl. A61M 16/10; A61M 16/12; A61M 16/20

(52) U.S. Cl. CPC A61M 16/1005; A61M 16/12; A61M 16/203; A61M 2016/1025

(57) ABSTRACT

A method and a control unit for executing the method. The method is a method for regulating a gas flow of at least one first gas to be admixed to at least one second gas. The method comprises at least one method step of a control of a gas valve. At least one manipulated variable for the control of the gas valve is determined from at least one correction regulator component and at least one feedforward component, the input variable of the feedforward component being a predicted gas flow setpoint value of the first gas.

### CROSS-REFERENCE TO RELATED APPLICATIONS

The present application claims priority under 35 U.S.C. § 119 of German Patent Application No. 102022104938.8, filed Mar. 2, 2022, the entire disclosure of which is expressly incorporated by reference herein.

### BACKGROUND OF THE INVENTION

### 1. Field of the Invention

The invention relates to a control unit and a method for regulating the gas flow of a first gas to be admixed to a second gas.

### 2. Discussion of Background Information

In many devices, in particular in medical devices, it is provided that two gases are mixed with one another. It can sometimes occur that the second gas is a gas mixture, which already comprises a specific concentration of the first gas. In general, the flow and thus the amount of at least one of the gases to be mixed (or gas mixtures) is regulated or generated by a valve. In general, a control loop or control circuit is used for this purpose, according to which the valve is controlled on the basis of the concentration of the first gas, for example oxygen, in the resulting mixture. The measurement of the oxygen concentration is generally inaccurate and slow, however. Problems result here, for example, in the supply of patients with the correct amount of oxygen at the correct time. Delays also occur in setting the correct concentration.

In view of the foregoing, it would be advantageous to have available a method and a device for efficient regulation of the gas composition.

### SUMMARY OF THE INVENTION

The invention provides a control unit for a ventilator, the control unit being configured and designed to execute a method for regulating a gas flow of at least one first gas to be admixed to at least one second gas, wherein the method comprises at least one step of controlling a gas valve, wherein at least one manipulated variable for the control of the gas valve is determined from at least one correction regulator component and at least one feedforward component, wherein the input variable of the feedforward component is a predicted gas flow setpoint value of the first gas.

In some embodiments, the control unit is characterized in that the method furthermore comprises: a first step of the overall flow prediction; a second step for flow scaling; a third step for determining a predicted gas flow setpoint value and/or a scaled predicted gas flow setpoint value.

In some embodiments, the control unit is characterized in that in the first method step, proceeding from a set pressure value and a ventilation situation, a predicted total flow setpoint value of the gas mixture of the at least two gases is determined. In some embodiments, the control unit is characterized in that the first method step comprises a patient flow model, wherein the set pressure value and the ventilation situation are at least partially incorporated in the patient flow model and wherein the result of the patient flow model and at least partially the ventilation situation are incorporated in a calculation of the predicted total flow setpoint value. In some embodiments, the control unit is characterized in that in the first method step, starting values for the patient flow model are determined via a patient model, in particular in the form of an RC equivalent model, from the ventilation situation.

In some embodiments, the control unit is characterized in that in the second method step, a scaled predicted total flow setpoint value and/or the scaled predicted gas flow setpoint value is calculated from the predicted total flow setpoint value alone or from the predicted total flow setpoint value together with the predicted gas flow setpoint value. In some embodiments, the control unit is characterized in that in the second method step, a scaled predicted total flow setpoint value and/or the scaled predicted gas flow setpoint value is calculated from the predicted gas flow setpoint value. In some embodiments, the control unit is characterized in that the second method step comprises scaling, wherein separate scaling factors and/or scaling functions are determined for the inspiration and expiration and a switch is made via a switch between the scaling factors and/or scaling functions depending on inspiration or expiration. In some embodiments, the control unit is characterized in that at least one comparison between a provided inspiration or expiration volume and an actually applied inspiration or expiration volume is taken into consideration for determining the scaling factors and/or the scaling functions.

In some embodiments, the control unit is characterized in that the third method step comprises a calculation of the predicted gas flow setpoint value from the predicted total flow setpoint value. In some embodiments, the control unit is characterized in that the third method step comprises the determination of a mean concentration mcO2% of the first gas in the second gas, wherein an exhalation volume is also incorporated in the determination of the mean concentration mcO2% of the first gas. In some embodiments, the control unit is characterized in that in the third method step, the determined mean concentration mcO2% of the first gas is also incorporated in the determination of the predicted gas flow setpoint value.

In some embodiments, the control unit is characterized in that the input variable for the feedforward component for determining the manipulated variable for controlling the gas valve is the scaled predicted gas flow setpoint value, optionally with consideration of the exhalation volume.

In some embodiments, the control unit is characterized in that the input variable for the correction regulator component comprises at least one parameter which describes a deviation of a gas flow actual value determined by at least one flow sensor from a predetermined gas flow setpoint value.

In some embodiments, the control unit is characterized in that the correction regulator component of the manipulated variable for controlling the gas valve becomes zero when the gas flow value corresponds to the gas flow setpoint value.

In some embodiments, the control unit is characterized in that the first gas is oxygen and the second gas is ambient air or compressed air/pressurized air or a gas mixture made up of ambient air and/or compressed air/pressurized air and/or an at least partially exhaled respiratory gas.

In some embodiments, the control unit is characterized in that the control unit is designed to calculate the exhalation volume on the basis of measurement data of at least one flow sensor.

In some embodiments, the control unit is characterized in that in the first method step a predicted total flow setpoint value is calculated from at least one ventilation situation, a set pressure value, and at least partially via a patient flow model; in the second method step, a scaled predicted total flow setpoint value is determined from the predicted total flow setpoint value and inspiration data and expiration data via scaling, wherein the scaling is adapted to the respective respiration phase; in the third method step, a scaled predicted gas flow setpoint value is determined from the scaled predicted total flow setpoint value and a mean concentration mcO2% of the first gas determined using an exhalation volume; in a fourth method step, the input variable for the feedforward component is the scaled predicted gas flow setpoint value.

The invention also provides a ventilator comprising at least one control unit as described above.

The invention furthermore provides a method for regulating a gas flow of at least one first gas to be admixed to at least one second gas, the method comprising at least one method step of a control of a gas valve, wherein at least one manipulated variable for the control of the gas valve is determined from at least one correction regulator component and at least one feedforward component. The method is characterized in that the input variable of the feedforward component is a predicted gas flow setpoint value of the first gas.

In some embodiments, the method is characterized in that it comprises at least one method step of the total flow prediction; one method step for flow scaling; one method step for determining a predicted gas flow setpoint value and/or scaled predicted gas flow setpoint value; and one method step for the control of the gas valve.

In some embodiments, the method is characterized in that it comprises at least one method step of the total flow prediction, wherein a predicted total flow setpoint value of the gas mixture of the at least two gases is determined starting from a set pressure value and a ventilation situation.

In some embodiments, the method is characterized in that the method step of the total flow prediction comprises a patient flow model, wherein the set pressure value and the ventilation situation are at least partially incorporated in the patient flow model and wherein the result of the patient flow model and at least partially the ventilation situation are incorporated in the calculation of the predicted total flow setpoint value.

In some embodiments, the method is characterized in that in the method step, starting values for the patient flow model are determined via a patient model from the ventilation situation. In some embodiments, the method is characterized in that the patient model in the method step is an RC equivalent model.

In some embodiments, the method is characterized in that the method comprises at least one method step for flow scaling, wherein in the method step for flow scaling, a scaled predicted total flow setpoint value and/or a scaled predicted gas flow setpoint value is calculated from the predicted total flow setpoint value and/or a predicted gas flow setpoint value.

In some embodiments, the method is characterized in that the method step comprises scaling, wherein separate scaling factors and/or scaling functions are determined for the inspiration and expiration and a switch is made via a switch between the scaling factors and/or scaling functions depending on inspiration or expiration. In some embodiments, the method is characterized in that at least one comparison between the provided inspiration or expiration volume and the actually applied inspiration or expiration volume is taken into consideration for determining the scaling factors and/or scaling functions.

In some embodiments, the method is characterized in that the method step for determining a predicted gas flow setpoint value comprises the calculation of a predicted gas flow setpoint value from a predicted total flow setpoint value.

In some embodiments, the method is characterized in that the method step comprises the determination of a mean concentration mcO2% of the first gas in the second gas, wherein an exhalation volume is also incorporated in the determination of the mean concentration mcO2% of the first gas. In some embodiments, the method is characterized in that in the method step, the determined mean concentration mcO2% of the first gas is also incorporated in the determination of the predicted gas flow setpoint value.

In some embodiments, the method is characterized in that in the method step, the scaled predicted gas flow setpoint value is determined.

In some embodiments, the method is characterized in that in the method step, the input variable for the feedforward component for determining the manipulated variable for controlling the gas valve is the scaled predicted gas flow setpoint value, optionally with consideration of the exhalation volume V Ruck.

In some embodiments, the method is characterized in that the input variable for the correction regulator component comprises at least one parameter which describes the deviation of the gas flow value from the gas flow setpoint value.

In some embodiments, the method is characterized in that the correction regulator component of the manipulated variable for controlling the gas valve becomes zero when the gas flow value corresponds to the gas flow setpoint value.

In some embodiments, the method is characterized in that the method step for flow scaling and the method step for determining a predicted gas flow setpoint value are exchangeable, wherein in the method step, the predicted gas flow setpoint value is calculated when the method step follows the method step or the scaled predicted gas flow setpoint value is determined if the method step follows the method step.

In some embodiments, the method is characterized in that the first gas is oxygen and the second gas is ambient air or compressed air/pressurized air and/or a gas mixture made up of ambient air and/or compressed air/pressurized air and/or an at least partially exhaled respiratory gas.

The invention furthermore provides a method for regulating the gas flow of at least one first gas to be admixed to at least one second gas, wherein the method comprises at least one method step of a control of a gas valve, wherein at least one manipulated variable is determined for the control of the gas valve from at least one correction regulator component and at least one feedforward component. The method is furthermore characterized in that
a. in a first method step, a predicted total flow setpoint value is calculated from at least one ventilation situation, a set pressure value, and at least partially via a patient flow model;
b. in a second method step, a scaled predicted total flow setpoint value is determined from the total flow setpoint value and inspiration data and expiration data via scaling, wherein the scaling is adapted to the respective respiration phase;
c. in a third method step, a scaled predicted gas flow setpoint value is determined from the scaled predicted total flow setpoint value and a mean concentration mcO2% of the first gas determined using an exhalation volume V_Rück;
d. in a method step for the control of the gas valve, wherein the input variable for the feedforward component is the scaled predicted gas flow setpoint value from the third method step.

The control unit is configured and designed to execute a method for regulating a gas flow of at least one first gas to be admixed to at least one second gas, wherein the method comprises at least one method step of a control of a gas valve, wherein at least one manipulated variable for the control of the gas valve is determined from at least one correction regulator component and at least one feedforward component, wherein the input variable of the feedforward component is a predicted gas flow setpoint value of the first gas.

In some embodiments, the control unit is characterized in that the control unit comprises a calculation unit, wherein the calculation unit is configured and designed to determine the predicted gas flow setpoint value of a first gas from a predicted total flow setpoint value.

In some embodiments, the control unit is characterized in that the calculation unit is configured and designed to determine the predicted total flow setpoint value from a set pressure value and a ventilation situation.

The invention also provides a ventilator, wherein the ventilator comprises a gas valve and a control unit, wherein the control unit is configured and designed to execute the above-described method.

In some embodiments, the ventilator is characterized in that the ventilator comprises at least one flow sensor, wherein the control unit is configured and designed to calculate the exhalation volume V_Rück on the basis of the measurement data of at least one flow sensor.

In some embodiments, the ventilator is characterized in that the control unit is configured and designed to determine the correction regulator component of the manipulated variable for controlling the gas valve from a comparison of a predetermined gas flow setpoint value and a gas flow value of the gas flow of the first gas determined by a flow sensor.

In some embodiments, the ventilator is characterized in that the control unit is configured and designed to control the gas valve on the basis of a manipulated variable, wherein the manipulated variable consists of a feedforward component and a correction regulator component and wherein the feedforward component has as the input variable the predicted gas flow setpoint value of the first gas.

In some embodiments, the ventilator is characterized in that the ventilator and/or the control unit comprises a calculation unit, wherein the calculation unit is configured and designed to determine the predicted gas flow setpoint value of a first gas from a predicted total flow setpoint value.

In some embodiments, the ventilator is characterized in that the calculation unit is designed to determine the predicted total flow setpoint value from a set pressure value and a ventilation situation.

In some embodiments, the ventilator is characterized in that the calculation unit is configured and designed to incorporate a patient flow model in the determination of the predicted total flow setpoint value.

In some embodiments, the ventilator is characterized in that the calculation unit is configured and designed to calculate a scaled predicted total flow setpoint value and/or a scaled predicted gas flow setpoint value from the predicted total flow setpoint value.

In some embodiments, the ventilator is characterized in that the calculation unit is configured and designed to scale the predicted total flow setpoint value depending on the respiration phase.

In some embodiments, the ventilator is characterized in that the calculation unit also incorporates a mean ambient air concentration of the first gas in the calculation of the scaled predicted gas flow setpoint value, wherein the mean ambient air concentration of the first gas is determined, inter alia, via an exhalation volume V_Rück.

In some embodiments, the ventilator is characterized in that the calculation unit is configured and designed to calculate the exhalation volume V_Rück on the basis of the measurement data of at least one flow sensor.

In some embodiments, the ventilator is characterized in that the calculation unit is configured and designed to determine the correction regulator component of the manipulated variable for controlling the gas valve from a comparison of a predetermined gas flow setpoint value and a gas flow value of the gas flow of the first gas determined by a flow sensor.

In some embodiments, the control unit is designed and configured to execute a method for regulating the gas flow of at least one first gas to be admixed to at least one second gas, wherein the method comprises at least one method step of a control of a gas valve, wherein at least one manipulated variable for the control of the gas valve is determined from at least one correction regulator component and at least one feedforward component, wherein
a. in a first method step, a predicted total flow setpoint value is calculated from at least one ventilation situation, a set pressure value, and at least partially via a patient flow model;
b. in a second method step, a scaled predicted total flow setpoint value is determined from the total flow setpoint value and inspiration data and expiration data via a scaling, wherein the scaling is adapted to the respective respiration phase;
c. in a third method step, a scaled predicted gas flow setpoint value is determined from the scaled predicted total flow setpoint value and a mean concentration of the first gas mcO2% determined using an exhalation volume V_Rück;
d. the method step is the fourth method step, wherein the input variable for the feedforward component is the scaled predicted gas flow setpoint value from the third method step.

The presented controller, the presented method, and the presented ventilator are conceived to achieve a total concentration of a specific gas in a gas mixture. For this purpose, a first gas is admixed to a second gas or gas mixture, wherein the second gas/gas mixture contains a concentration of the first gas. This is achieved by deliberate control of a gas valve which generates a flow of the first gas which is admixed to the second gas. In some embodiments, it is provided that the gas mixture made up of first and second gas can at least partially flow back. It can be provided here that the concentration of the first gas in the gas flowing back is also taken into consideration and the control of the gas valve for the flow of the first gas is adapted accordingly.

It is to be noted that the features listed individually in the claims can be combined with one another in any technically reasonable manner and disclose further embodiments of the invention. The description additionally characterizes and specifies the invention in particular in conjunction with the figures.

It is furthermore to be noted that a conjunction “and/or” used herein, which stands between two features and links them to one another, is always to be interpreted so that in a first embodiment of the subject matter according to the invention, only the first feature can be provided, in a second embodiment only the second feature can be provided, and in a third embodiment both the first and the second feature can be provided.

A ventilator is to be understood as any device which assists a user or patient in natural respiration, takes over the ventilation of the user or living being (e.g., patient and/or newborn baby and/or premature baby) and/or is used for respiratory therapy and/or influences the respiration of the user or patient in another manner. These include, for example, but not exclusively, CPAP and bilevel devices, narcosis or anesthesia devices, respiratory therapy devices, (clinical, nonclinical, or emergency) ventilators, high flow therapy devices, and coughing machines. Ventilators can also be understood as diagnostic devices for ventilation. Diagnostic devices can generally be used here for detecting medical and/or respiration-related parameters of a living being. These also include devices which can detect and optionally process medical parameters of patients in combination with respiration or exclusively relating to respiration.

If not expressly described otherwise, a patient interface can be understood as any peripheral device which is designed for the interaction of a ventilator with a living being, and vice versa, in particular for therapeutic, ventilation and/or diagnostic purposes. In particular, a patient interface can be understood as a mask of a ventilator or a mask connected to the ventilator. This mask can be a full face mask, thus enclosing the nose and mouth, or a nose mask, thus a mask only enclosing the nose. Tracheal tubes or cannulas and so-called nasal glasses can also be used as a mask or patient interface. In some cases, the patient interface can also be a simple mouthpiece, for example a tube, through which the living being at least exhales and/or inhales.

The connection between patient interface and ventilator can be produced via a variety of different connecting or hose systems. For example, a leak system can be provided, in which exhaled respiratory gas can escape through a deliberate leak in the patient interface and/or the ventilation hose. A 2-hose system can also be provided, in which the exhaled respiratory gas is returned through an expiration hose into the ventilator, and the exhalation volume is possibly measured there and can escape through the ventilator into the ambient air. A valve essentially prevents the exhaled respiratory gas from entering the inspiration hose here. It can also be provided that the patient interface and the ventilator are connected via a 1-hose system having patient valve, which is controlled, for example, via the ventilator. At least during the expiration, the valve can be switched so that the exhalation air can escape directly from the hose system into the ambient air.

The terms “gas” and “gas mixture” are used synonymously in the description and the claims. In particular, a gas can also describe a gas mixture. In particular, the second gas can be a gas mixture. The second gas can be, for example, the ambient air, which is aspirated, or also compressed air/pressurized air, which is supplied from gas cylinders or a supply line. In some embodiments, the second gas can also be a mixture of the aspirated ambient air or the compressed air/pressurized air together with a gas mixture that is exhaled or is flowing back.

The gas mixture flowing back can have a concentration of the first gas here which deviates from the originally generated gas mixture. In some embodiments, the first gas is oxygen and the second gas is ambient air and/or a gas mixture flowing back and/or a mixture thereof.

It is furthermore to be noted that “predicted gas flow setpoint value” is to be understood as any type of predicted gas flow setpoint value, if not clearly described otherwise. In particular, “predicted gas flow setpoint value” can also comprise a scaled predicted gas flow setpoint value in addition to the (unscaled) predicted gas flow setpoint value. In addition, “predicted total flow setpoint value” is to be understood as any type of predicted total flow setpoint value if not clearly described otherwise. In particular, “predicted total flow setpoint value” can also comprise a scaled predicted total flow setpoint value in addition to the (unsealed) predicted total flow setpoint value.

The invention follows the basic concept of replacing the input variable for the feedforward component of the manipulated variable. In a classic feedforward regulation for the gas flow, at least one input variable for the feedforward component is a gas flow setpoint value calculated from a measured total flow. This gas flow setpoint value calculated from a measured total flow is replaced by a predicted gas flow setpoint value, which is determined from a predicted total flow.

According to the method according to the invention, which can be executed, for example, by the ventilator according to the invention, the concentration of a first gas, for example oxygen, is regulated, inter alia, by a feedforward regulation of the gas flow of the first gas. The goal of the feedforward regulation is to predetermine the gas flow and regulate it accordingly to the next or current breath. In addition to the feedforward component, a correction regulator component, similar to a feedback, is provided. The correction regulator component is based here on a comparison of the actually measured gas flow to the provided value in order to thus correct possible inaccuracies or deviations, independently of the error source. If a gas flow occurs due to the feedforward component which corresponds precisely to the set target value, no correction regulation takes place. Systematic errors of the feedforward regulation can also be compensated for by the correction regulation, for example. If the resulting gas flow from the feedforward regulation always deviates by a certain percentage from the predetermined target value, for example, this can be compensated for by the correction regulation.

A predicted gas flow setpoint value of the gas to be regulated is used as the input variable for determining the feedforward component. In a first method step, a total flow setpoint value of the respiratory gas is predicted to determine the predicted gas flow setpoint value. The current ventilation situation is also incorporated here in the prediction. On the one hand, the ventilation situation can reflect settings/parameters for the ventilation of the patient and, on the other hand, patient-related factors, for example the physiology of the patient, can also be incorporated. In addition to the current ventilation situation, a patient flow model is also incorporated in the calculation of the total flow prediction. Input variables and/or factors for the patient flow model are at least a part of the current ventilation situation and the predetermined (ventilation) pressure. In order to be incorporated in the patient flow model, the current ventilation situation is converted, for example, via an RC equivalent model.

In some embodiments, it is provided that inaccuracies occurring due to the activation of the gas valve can also be compensated for by the regulation or control of the gas flow to set the gas concentration of the first gas. For this purpose, a correction factor is determined in a further method step, which can be applied to the total flow prediction, for example. This correction factor can also be adapted, for example, to the respiration phase of the patient—thus inspiration and expiration. To determine the correction factor, it can be provided that, optionally averaged over multiple breaths, a ratio is developed between a volume specification and/or an expected volume and the actually measured volume. On the basis of this ratio, a correction factor is determined for the respective respiration phase, which is applied to the total flow prediction.

It can be provided that the predicted gas flow setpoint value of the first gas to be regulated is determined on the basis of the total flow prediction computed using the correction factor. In some embodiments, it can also be provided that initially the predicted flow value of the gas to be regulated is determined from the total flow prediction and this predicted flow value is then scaled via the correction factor.

In some embodiments, it is provided in one method step that optionally a volume part exhaled into the ventilator and/or pushed back by the exhalation of the patient is also taken into consideration. The exhaled volume component is also incorporated here in the determination of the predicted flow value of the gas to be regulated. This method step can take place in parallel to, before, or after the method step of determining the correction factor. In some embodiments, it is provided that the method step of taking into consideration the exhalation volume takes place after the method step of the determination of the correction factor.

In one embodiment, the method sequence is provided so that in a first method step a total flow prediction is carried out. In a second method step, it is then provided that a correction factor is determined, which is combined by calculation with the total flow prediction. In a third method step, an optional determination of the exhalation volume takes place. The determination of the predicted gas flow of the gas to be regulated then takes place, optionally with incorporation of the exhalation volume. The predicted gas flow of the gas to be regulated is then used as the input variable for the feedforward regulation of the gas flow. Together with a correction regulation, a control signal for the valve switching is generated, by which the actual gas flow is regulated or generated. The measured actual gas flow is a variable which is used for the correction regulation here.

### BRIEF DESCRIPTION OF THE DRAWINGS

The invention will be described in more detail by way of example on the basis of FIGS. 1 to 6, in which:

FIG. 1 is a schematic flow diagram of an exemplary embodiment of a method of the present invention;

FIG. 2 schematically shows an exemplary sequence of method step 110 in FIG. 1;

FIG. 3 schematically shows an exemplary sequence of method step 120 in FIG. 1;

FIG. 4 schematically shows an exemplary sequence of method step 130 in FIG. 1;

FIG. 5 schematically shows an exemplary sequence of method step 140 in FIG. 1; and

FIG. 6 schematically shows an exemplary embodiment of a ventilator of the invention.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

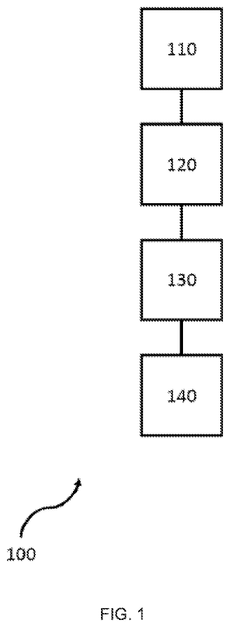

The sequence of the method 100 for regulating the gas flow 146 is shown in FIG. 1 in a schematic overview of an exemplary embodiment. The method 100 comprises a method step 110 of the total flow prediction, a method step 120 of the flow scaling, a method step 130 of the determination of a scaled predicted gas flow setpoint value 133 with optional consideration of the exhalation volume, and a method step 140 of the control of the gas valve. Method step 140 of the control of the gas valve also comprises the determination of the manipulated variable for the control of the gas valve from a correction regulator component and a feedforward component.

Method 100 for regulating the gas flow 146 is based on a feedforward regulation, wherein the input variable of the feedforward component is replaced by the manipulated variable for controlling the gas valve. The advantage of a feedforward regulation here is that the regulation passes by a pure control, so that an enhanced reaction time can be achieved, for example in the event of gas flow changes. In a classic feedforward regulation for the gas flow 146, at least one input variable for the feedforward component can be a gas flow setpoint value 141 calculated from a measured total flow. This gas flow setpoint value 141 calculated from a measured total flow is replaced by a predicted gas flow setpoint value, which is ascertained from a predicted total flow setpoint value 116. The determination of the predicted total flow setpoint value 116 is carried out here, for example, in method step 110 of the total flow prediction. In method steps 120, 130, the predicted gas flow setpoint value is determined from the predicted total flow setpoint value 116 and scaled to arrive at a scaled predicted gas flow setpoint value 133. In some embodiments, the predicted total flow setpoint value 116 is initially scaled and in method step 130, the scaled predicted gas flow setpoint value 133 is determined from the scaled predicted total flow setpoint value 127. Alternatively or additionally, the predicted gas flow setpoint value is determined directly from the predicted total flow setpoint value 116, which is then scaled accordingly in method step 130. If the optional consideration of the exhalation volume V_Rück is provided, V_Rück is already incorporated in the calculation of the predicted gas flow setpoint value before the scaling. Optionally, in method step 130 of the determination of the scaled predicted gas flow setpoint value 133, the exhalation volume V_Rück is also incorporated to arrive at the correspondingly adapted gas flow setpoint value 133. The predicted gas flow setpoint value 133 is used in method step 140 as an input variable for the feedforward component of the manipulated variable for the control of the gas valve.

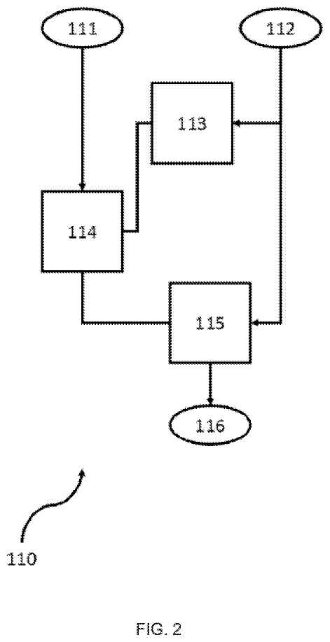

Method step 110 for the total flow prediction, in which the predicted total flow setpoint value 116 is determined, is schematically shown in an exemplary embodiment in FIG. 2.

FIG. 2 schematically shows an exemplary sequence of method step 110 for the total flow prediction. Inter alia, the input variables for the total flow prediction are the pressure 111 specified for ventilation and the current ventilation situation 112. On the one hand, the ventilation situation 112 can reflect here settings/parameters for the ventilation of the patient and, on the other hand, patient-related factors, for example the physiology of the patient, can also be incorporated. Settings/parameters for ventilation can be, for example, pressures (IPAP, EPAP, PEEP, etc.), flows, volumes, ventilation times, ventilation mode (CPAP, APAP, BiLevel, etc.), respiration frequencies, etc. Patient-related factors, for example the physiology of the patient, are, for example, age, height, weight, sex, illnesses, etc.

One possibility for describing the current ventilation situation 112 is the modeling of the patient via an RC equivalent model 113. In addition to an RC equivalent model, other and/or further patient models can also be used, which describe the patient more accurately, for example. The parameterization of the patient flow model 114 takes place, in that, inter alia, the parameters of the preceding breaths are assessed. The course of the patient parameters is estimated or determined from the pressure and flow, for example, via the RC equivalent model 113. Together with at least the predetermined pressure 111, it is determined in a patient flow model 114, for example, which gas flow is to arrive at the patient. Via the exemplary RC equivalent model 113, for example, the patient is thus initially represented as a starting point and in the following patient flow model 114, inter alia, a patient flow is determined together with the set ventilation pressure 111. For example, the parameters of the next-to-last breath are used to assess the last breath in the interaction between the RC equivalent model 113 and the patient flow model 114.

The predicted total flow 116 is calculated via a calculation 115 from the calculations of the patient flow model 114 and the current ventilation situation 112. The parameters/factors of the current ventilation situation 112, which can be incorporated in the patient flow model and directly in the calculation 115 of the predicted total flow 116, can be identical or different here. It can be provided that some parameters of the current ventilation situation 112 are incorporated both in the patient flow model 114 and also directly in the calculation 115 of the predicted total flow 116. Some parameters of the current ventilation situation 112 can also only be incorporated directly into the patient flow model 114 or the calculation 115.

The predicted total flow setpoint value 116 reflects the total flow which is conveyed to/from the patient. Thus, for example, the flow of the aspirated ambient air plus the gas flow 146.

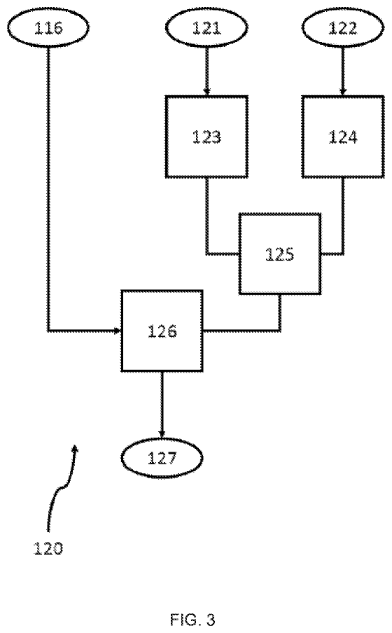

Method step 120 of the flow scaling is schematically shown in an exemplary embodiment in FIG. 3. In a scaling step 126, the predicted total flow setpoint value 116 is scaled. For this purpose, inspiration data 121 and expiration data 122 of patient 212 are used as a further input variable for the scaling 126. In calculation steps 123, 124, which are each carried out individually for the inspiration and expiration, a scaling factor or a scaling function is determined, using which the predicted total flow setpoint value 116 is scaled. A switch is made here via switch 125 depending on the respiration phase between scaling for the inspiration or expiration.

The inspiration data 121 or expiration data 122 are, for example, calculated values which take into consideration at least a ratio between a provided inspiration or expiration volume and the actually applied inspiration or expiration volume. This ratio is optionally averaged over multiple breaths and possibly weighted. A weighting can be carried out, for example, on the basis of the current situation of the breaths, for example more recent breaths are weighted higher than breaths which are farther in the past. In some embodiments, the determination of the scaling factor or the scaling function can also take into consideration further data and influences.

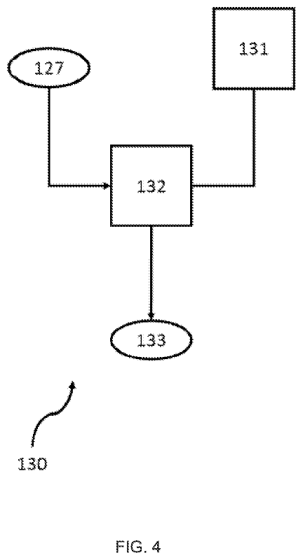

An exemplary embodiment of method step 130 for determining the scaled predicted gas flow setpoint value 133 is schematically shown in FIG. 4. In the exemplary embodiment, method step 130 also comprises the optional consideration of the exhalation volume V_Rück. The exhalation volume V_Rück is incorporated, for example, via the determination 131 of a mean concentration mcO2% of the first gas (for example oxygen) in the second gas/gas mixture in the calculation 132 of the scaled predicted gas flow setpoint value 133. The mean concentration mcO2% of the first gas in the second gas may be calculated in simplified form on the basis of the example oxygen via the formula:

$$mc O 2 \% = \frac{( c O 2 R ü ck \cdot {V}_{R ü ck} ) + ( ( {V}_{Ges} - {V}_{R ück} ) \cdot 21 \% )}{{V}_{G e s}}$$

wherein cO2Rück is the oxygen concentration in the exhalation volume V_{Rück} and V_{Ges} is the total respiration volume of the last breath. It is to be noted in this case that the second gas is, for example, a gas mixture, which can comprise a specific concentration of the first gas. The gas mixture (second gas) of respiratory gas can be given as an example here, which contains a specific concentration of oxygen (first gas). The mean concentration mcO2% takes into consideration here both the freshly aspirated ambient air and also the exhaled respiratory gas. The goal of the observation of the exhalation volume V_{Rück} is that a total concentration of the first gas in the second gas can be set. The concentrations of the first gas in the first gas (100%) and the second gas are at least approximately known here. The exhalation, thus the flowing back of the generated gas mixture made up of first and second gas, changes the concentration of the first gas in the gas mixture here, to which the first gas is to be admixed. Instead of the measured oxygen concentration cO2Rück, the target concentration of the first gas (here: oxygen) in the second gas (here: the respiration gas) can also be used here. The specified 21% relates to the oxygen content of the ambient air. Alternatively or additionally, it can also be provided that the oxygen content of the aspirated ambient air is measured and the measured value is replaced instead of the specified 21%. If it is provided that the flow of a gas other than oxygen is to be regulated via the proposed method, the values are to be adapted accordingly. An additional or alternative approach can also be that a concentration of the first gas which is not constant, but rather is high and then drops is to be used as the target variable. The concentration can thus be lower in the exhalation volume, for example, and in the next breath the first gas can be admixed in accordance with the target concentration.

At least the target concentration of the first gas in the respiratory gas is also incorporated in the calculation 132 of the scaled predicted gas flow setpoint value 133, for example, in addition to the mean ambient air concentration mcO2% and the scaled predicted total flow setpoint value 127.

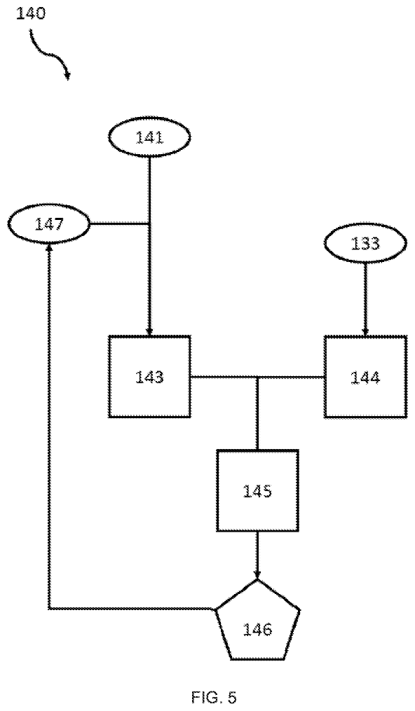

Method step 140 for the control 145 of the gas valve is schematically shown in an exemplary embodiment in FIG. 5. The exemplary embodiment in FIG. 5 also comprises the determination of the manipulated variable for the control 145 of the gas valve 203 from the correction regulator component 143 and the feedforward component 144. One of the input variables for the feedforward component 144 is in this case the scaled predicted gas flow setpoint value 133, which is determined, for example, in method step 130.

For the correction regulation component 143, the real gas flow actual value 147 is matched with the predetermined gas flow setpoint value 141. If the real gas flow actual value 147 corresponds to the predetermined gas flow setpoint value 141, the correction regulator component 143 becomes zero, therefore no deviation is to be established. The correction regulator component 143 is independent here, for example, of the cause of a possible deviation, rather solely specifies whether and by how much the set gas flow setpoint value 141 and the real gas flow actual value 147 deviate from one another. In some embodiments, it can additionally be provided that an error analysis is carried out and the cause of the deviation is also incorporated in the correction regulator component 143. Alternatively or additionally, it can be provided that in the event of deviations between real gas flow actual value 147 and the set gas flow setpoint value 141, a cause-independent correction is performed via the correction regulator component 143, but an error analysis is also carried out simultaneously and this is taken into account in the feedforward component 144.

The feedforward component 144 is configured so that possible disturbances and/or inaccuracies of the valve activation can already be incorporated beforehand. In a regular feedforward regulation, the determination of the feedforward component 144 can be carried out on the basis of the predetermined gas flow setpoint value 141, which is in turn determined from a measured total flow. In the described method, the feedforward component 144, in contrast, is based on a predicted gas flow setpoint value, for example a scaled predicted gas flow setpoint value 133. The reaction time can be minimized and/or the regulation quality can be improved in this way, for example. The feedforward component 144 is determined here in accordance with the known prior art. The feedforward component 144 can be determined, for example, via a model, such as an inverse system model, of the valve to be controlled or a characteristic curve of the valve. In some embodiments, a mixture of the forms and/or other models and/or characteristic curves can also be provided for determining the feedforward component 144.

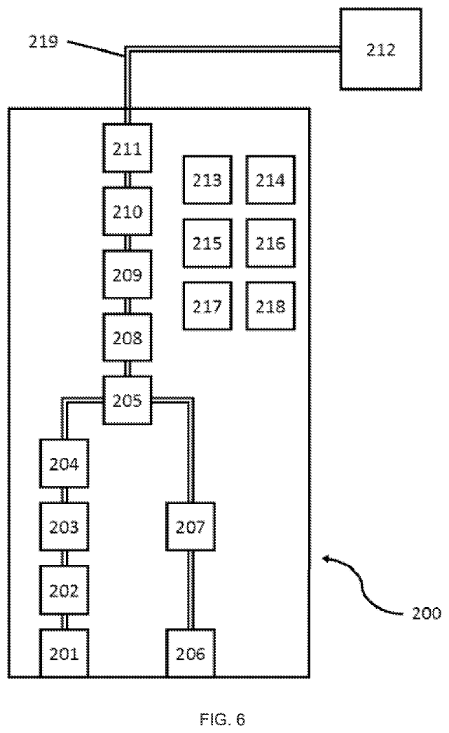

An exemplary embodiment of a ventilator 200 is shown schematically and greatly simplified in FIG. 6. The ventilator 200 is configured and designed here, in particular by the control unit 214 and the calculation unit 213, to control a gas valve 203 via a feedforward regulation.

Ventilator 200 is designed and configured to monitor and at least partially analyze the respiration of the patient 212. For example, the ventilator 200 is configured to recognize the respiration phases (inspiration, expiration). The ventilator 200 can also be configured to recognize various respiration situations, such as apnea, snoring, breathing interruptions, irregular breathing, weak breathing, etc. It can be provided that the ventilator 200 is configured to automatically set at least partially determined ventilation parameters such as pressure and/or flow and/or volume and/or frequency to assist and/or specify the respiration. The ventilator 200 is configured and designed to assist the patient 212 at least in phases in the respiration and/or to specify the respiration of the patient 212.

The ventilator 200 comprises at least two gas sources 201, 206. The gas sources 201, 206 are each configured to provide at least one gas or gas mixture to the ventilator 200.

In the exemplary embodiment shown, the gas source 201 represents an oxygen source. For example, the oxygen source 201 comprises at least one connection for a pressurized gas connection, such as an oxygen cylinder. It can alternatively or additionally also be provided that the oxygen source 201 comprises an oxygen concentrator which is integrated in or connected to the ventilator 200. If a pressurized gas source, for example an oxygen cylinder, is connected to the oxygen source 201 of the ventilator 200, it can thus be provided that a pressure reducer is arranged between the pressurized gas source and the ventilator 200. Alternatively or additionally, a pressure reducer from the oxygen source 201 can also be arranged in or on the ventilator 200. It can moreover optionally be provided that the oxygen source 201 comprises a filter.

A pressure sensor 202 is pneumatically connected to the oxygen source 201, for example, which is configured to measure the pressure of the supplied oxygen. In some embodiments, the ventilator 200 is configured and designed to draw a conclusion about the fill level of the oxygen source 201 or a pressurized gas source connected thereto on the basis of the measured pressure of the pressure sensor 202. In some embodiments, a warning or alarm is output via the user interface 218 in the event of a low fill level.

The pressure sensor 202 is followed by a controllable valve 203, via which the oxygen flow 106 from the oxygen source 201 to the mixing area 205 is regulated. The valve 203 is controlled, for example, via the control unit 214 on the basis of the method according to the invention.

A flow sensor 204 is arranged following valve 203, which is configured and designed to measure the flow of the oxygen flow.

The at least one second gas source 206 is designed in the embodiment shown by way of example as an intake area 206 for ambient air. The intake area 206 is optionally equipped with a filter, which filters the ambient air. For example, pathogens and other contaminants are thus at least partially filtered out of the ambient air. In some embodiments, the intake area 206 can comprise a gas delivery unit, which is designed to aspirate ambient air. In the embodiment shown by way of example in FIG. 5, the ambient air is aspirated by fan 208 via the intake area 206. Alternatively or additionally to the intake area 206, the ventilator 200 can also have a connection to a pressurized gas source, such as a pressurized air cylinder. A flow sensor 207 is arranged on the gas source 206 or following the intake area 206, which is configured and designed to measure the gas flow from the intake area 206 in the direction of the mixing area 205.

The ventilator 200 comprises a mixing area 205 in which the gas flow from the gas source 201 and from the gas source 206 come together and are at least partially mixed. In some embodiments, the mixing area 205 is designed as a mixing chamber, which has a volume and optionally has specific structures which improve mixing of the two gases. It can alternatively or additionally also be provided that active mixing is provided, for example by rotors and/or vortexes. In some embodiments, the mixing area 205 is implemented by simply bringing together the two gas paths from the gas sources 201, 206. For example, a Y-piece can be provided for this purpose.

A controllable fan 208 is arranged following the mixing area 205, which is designed and configured to convey the gas mixture, for example as a respiratory gas, out of the mixing area 205. It is to be noted here that in some situations the valve 203 is closed and no gas flows out of the gas source 201 into the mixing area 205, so that the gas mixture or the respiratory gas from the mixing area 205 exclusively consists in some circumstances of the aspirated gas/gas mixture from the gas source/the intake area 206. In some embodiments, Fan 208 is also configured and designed to aspirate ambient air via the intake area 206. Fan 208 is configured to provide a settable pressure and/or flow of respiratory gas—conveyed out of the mixing area 205.

The oxygen content of the conveyed respiratory gas is measured via oxygen sensor 209. The measured oxygen content can also, for example, be incorporated in the control of valve 203, for example to be able to correct deviations in relation to a setpoint value. For example, the ambient air can have a different oxygen concentration than the assumed 21%, so that the correct oxygen flow 116 is set, but the oxygen concentration in the respiratory gas nonetheless does not correspond to the setpoint value.

For example, a pressure sensor 210 is arranged following the oxygen sensor 209, which is configured to detect the respiratory gas pressure of the respiratory gas line after fan 208. For example, a flow sensor 211 is arranged following the pressure sensor 210, which is configured and designed to detect or measure the respiratory gas flow.

The ventilator 200 is connected by way of example via a hose system 219 and a patient interface (not shown) to patient 212. For example, a leak system or a 2-hose system or a 1-hose valve system can be used as the hose system 219. In a leak system, the hose system and/or the patient interface has a deliberate leak through which the respiratory gas exhaled by patient 212 can at least partially escape. A part of the exhaled respiratory gas is also conducted in the direction of the ventilator 200 here in some embodiments. It can be provided that the volume of the patient interface and the hose system are taken into consideration so that penetration of the exhaled respiratory gas into the ventilator 200 can be essentially prevented. In some embodiments of the ventilator 200, an additional valve, for example a check valve, is provided in the area of the connection of the hose system 219 to the ventilator 200, which essentially prevents breathing back into the ventilator 200. In a 1-hose valve system, for example, it is provided that a valve close to the patient is controlled by the ventilator 200 so that the exhalation air can escape directly through the valve into the surroundings at least temporarily during the expiration of the patient.

To use a 2-hose system, the ventilator 200 has separate inspiration and expiration branches (not shown). Respiratory gas is conveyed to patient 212 via the inspiration branch during the inspiration of the patient 212. The exhaled respiratory gas is conducted through ventilator 200 and conducted into the surroundings via the expiration branch during the expiration of the patient 212. In some embodiments, the exhaled respiratory gas is filtered via a filter in the expiration branch. Upon the use of a 2-hose system, for example, a valve is arranged in the hose system 219, which closes the inspiration branch during the expiration phase so that exhaled respiratory gas cannot penetrate into the inspiration branch. The exhaled respiratory gas is returned here through a second hose to an expiration branch into the ventilator 200 and conducted through it, for example, into the ambient air. For example, it can occur due to valve delays that a small part of the respiratory gas is pressed back within the inspiration branch of the ventilator 200. However, exhaled respiratory gas is essentially prevented here from reaching the inspiration branch of the ventilator 200.

For example, the ventilator 200 comprises at least one calculation unit 213, a control unit 214, a detection unit 215, a monitoring unit 216, a storage unit 217, and a user interface 218.

The calculation unit 213 is configured and designed to calculate the manipulated variable for controlling the gas valve 203 and/or the feedforward component 144 and/or the correction regulator component 143 and/or the input variable, for example a predicted gas flow setpoint value, for the feedforward component 144.

The calculation unit 213 is designed, for example, for the purpose of at least partially executing method 100. In some embodiments, the calculation unit 213 is configured to execute the calculation steps of the method 100, and the control unit 214 is configured to execute the control of the gas valve 203 based thereon. The calculation unit 213 is designed in a first method step 110 to forecast/predict a total flow setpoint value 116 from the current ventilation situation 112 and a set ventilation pressure 111 (pressure setpoint). In a further method step 120, the calculation unit 213 calculates a scaled total flow setpoint value 127 from the predicted total flow setpoint value 116 and inspiration and expiration data 12. From the scaled total flow setpoint value 127 and with optional incorporation of the exhalation volume V_Rück , the calculation unit determines in a further method step 130 a scaled predicted gas flow setpoint value 133.

In some embodiments, it can be provided that the calculation unit 213 first calculates in method step 130 a predicted gas flow setpoint value, optionally with incorporation of the exhalation volume V_Rück, from the predicted total flow setpoint value 116. This predicted gas flow setpoint value is then used in place of the total flow setpoint value 116 in method step 120 as an input value for the determination of a scaled gas flow setpoint value 133 (instead of a scaled total flow setpoint value 127). Method step 120 and method step 130 can thus be exchangeable, wherein depending on the sequence first a scaled total flow setpoint value 127 and then the scaled gas flow setpoint value 133 are determined or first a predicted gas flow setpoint value is calculated from the predicted total flow setpoint value 116, which is then scaled to obtain the scaled gas flow setpoint value 133.

The scaled predicted gas flow setpoint value 133 is then used in method step 140 as an input variable of the feedforward component 144 of the manipulated variable for controlling 145 the gas valve 203. The manipulated variable can be determined, for example, by the control unit 214 and/or by the calculation unit 213. The control unit 214 is configured here to control the gas valve 203 on the basis of the manipulated variable determined from the feedforward component 144 and a correction regulator component 143.

In some embodiments, it can be provided that in the calculation unit 213, method steps 110, 120, 130 and the determination of the feedforward component 144 are represented via a single calculation, wherein the respective input variables are used as variables. In some embodiments, it can also be provided that the one calculation also comprises the determination of the manipulated variable for control 145 of the gas valve 203. The method steps shown in FIGS. 1 to 5 can thus be carried out at least partially in a single calculation, wherein the individual method steps represent, for example, individual parts of an equation.

The correction regulator component 143 is based on a comparison of a set gas flow setpoint value 141 and the real measured gas flow actual value 147. The gas flow actual value 147 is, for example, measured by the flow sensor 204 and detected by the detection unit 215.

Control unit 214 is configured and designed to control at least the valve 203. In some embodiments, the control unit 214 is also configured to control the fan 208. Moreover, it can be provided that control unit 214 is designed and configured to control the entire ventilator 200. In some embodiments, it can alternatively or additionally be provided that separate control units are provided for different functions and/or modules and/or components.

It can be provided in some embodiments that the calculation unit 213 is at least partially integrated in the control unit 214 and/or the control unit 214 comprises the calculation unit 213.

The detection unit 215 is configured and designed to detect and possibly to process the values measured by the sensors. In some embodiments, the detection unit 215 is configured to receive and/or record a signal generated by the sensors, for example in the form of voltage and/or amperage and/or frequency. It can alternatively or additionally be provided that detection unit 215 is also designed to convert the signals of the sensors into values.

The monitoring unit 216 is configured and designed to detect (technical) problems of the ventilator 200. Technical problems can be, for example, a low battery level, faults in the electronics, a defective battery, a defective component and/or module, a power failure, an incorrectly functioning accessory part, an implausible measured value, or leaving a permitted temperature range. The monitoring unit 216 can furthermore be designed to generate an alarm and/or a report in the event of a recognized technical problem. In some embodiments, it can also be provided that monitoring unit 216 is designed to detect a low fill level and/or an inadequate supply with an operating material, such as a gas. For example, it can be provided that monitoring unit 216 is designed to detect on the basis of a pressure of the gas source 201 of the first gas whether the supply is decreasing and/or is inadequate.

The (intermediate) results of the calculation unit 213 and the measured values detected by the detection unit 215 are stored and/or temporarily stored in the storage unit 217. A temporary storage in this case means that the data are automatically erased or released to be overwritten after a certain time span and/or an (automatically) executed action and/or an event.

Via the user interface 218, for example a unit comprising input elements and output elements, data and/or items of information can be input and output. Settings for the ventilation and for the configuration of the ventilator 200 can be performed via the input elements, for example. The output element, for example a display, is configured to display items of information and data on the current ventilation and settings. Moreover, alarms and warnings with respect to the functionality of the device and also the ventilation of patient 212 can be output via the user interface 218. Specifications on the ventilation, e.g., pressures, flow, volume, duration, program, can be input, for example, via the user interface 218. An (initial) target value for the oxygen concentration in the respiratory gas can also be defined. In some embodiments, the target value of the oxygen concentration in the respiratory gas is optionally automatically adapted by the ventilator 200 during the ventilation. In some embodiments, it can moreover be provided that an initial oxygen concentration is defined by a user and the ventilator 200 automatically adjusts the oxygen concentration during use. For example, it can also be provided that an oxygen concentration is initially specified there and the ventilator 200 automatically sets the specified oxygen concentration only when, for example, the oxygen supply of the patient 212 can no longer be maintained by the preset value and/or the patient 212 is oversupplied to a harmful extent. For this purpose, for example, it can be provided that ventilator 200 is supplied with data on the oxygen saturation of the blood of the patient 212 via a pulse oximeter.

Threshold and limiting values, for example for minimum and maximum oxygen concentration, minimum and maximum oxygen flow, minimum and maximum respiratory gas pressures, etc., can also be input via the user interface, for example. The threshold and limiting values can also apply as alarm limits here.

In some embodiments, the user interface 218 alternatively or additionally comprises an interface for connecting external display and/or input devices, such as display screens, keyboards, and/or remote controls.

It is to be noted that in some embodiments in addition to the components shown of the ventilator 200, further elements can also be arranged in the ventilator 200 and/or can be connected thereto. For example, an additional respiratory gas humidifier, optionally with respiratory gas heating, can be provided. It can also be provided, for example, that a nebulizer is arranged in the ventilator 200 and/or is connected to the ventilator 200, for example. In some embodiments, an expiration branch together with corresponding valves and controller can additionally be provided. The respiratory gas exhaled by patient 212 is conducted in the ventilator 200 to the ambient air via such an expiration branch. Moreover, one or more bypass valves can optionally also be arranged in the pneumatics of the ventilator 200, for example, in order to enable respiration for the patient at least temporarily in the event of a lack of power supply.

If additional components are connected to the ventilator 200 and/or arranged in the ventilator 200, it can be provided that these are also incorporated by the calculation unit 213 in the determination of a predicted gas flow setpoint value. For example, a change of the gas flow due to the use of a nebulizer and/or respiratory gas humidifier can be taken into consideration.

Moreover, it can also be provided that the ventilator 200 has at least one interface for connection to distant remote stations, for example for telemedicine.

In some embodiments, it can also be provided that the ventilator 200 is embodied as an anesthesia device. For this purpose, at least one, preferably additional gas source is provided so that anesthesia gas is introduced into the respiratory gas via this gas source. For this purpose, it can also be provided that the controller according to the invention is used to control the flow of the anesthesia gas. For example, it can be provided that the controller according to the invention is used in parallel for the control of the gas valve 203 for the first gas source 201 and also for the control, for example, of a further gas valve of the anesthesia gas source.

In some embodiments, it can also be provided that the control unit 214 is used separately, thus independently of a ventilator 200, to control a gas valve 203. For example, it can also be provided that control unit 214 is used for the control of a gas valve 203 in other contexts, in which mixing of at least two gases is provided. This applies accordingly to the method. In this case, for example, the patient model 114 is replaced by a model of a consumer. The inspiration can be viewed, for example, as a supply of the gas mixture to the consumer, wherein the expiration can correspond to an expulsion of the consumed or converted gas mixture, for example.

### LIST OF REFERENCE NUMERALS

100 method
110 first method step
111 pressure (predetermined)
112 ventilation situation
113 RC equivalent model
114 patient flow model
115 calculation of the predicted total flow setpoint value
116 total flow setpoint value (predicted)
120 second method step
121 inspiration data
122 expiration data
123 calculation step
124 calculation step
125 switch
126 scaling
127 total flow setpoint value (predicted, scaled)
130 third method step
131 determination (mcO2%)
132 calculation of the predicted gas flow setpoint value
133 gas flow setpoint value (predicted)
134 gas flow setpoint value (predicted, scaled)
140 method step
141 gas flow setpoint value (predetermined)
143 correction regulator component
144 feedforward component
145 control
146 gas flow
147 gas flow actual value
200 ventilator
201 gas source
202 pressure sensor
203 gas valve
204 flow sensor
205 mixing area
206 gas source/intake area
207 flow sensor
208 fan
209 oxygen sensor
210 pressure sensor
211 flow sensor
212 patient
213 calculation unit
214 control unit
215 detection unit
216 monitoring unit
217 storage unit
218 user interface
219 hose system

What is claimed is:

1. A control unit for a ventilator, wherein the control unit is configured and designed to execute a method for regulating a gas flow of at least one first gas to be admixed to at least one second gas, wherein the method comprises at least one step of a control of a gas valve, wherein at least one manipulated variable for the control of the gas valve is determined from at least one correction regulator component and at least one feedforward component, an input variable of the feedforward component being a predicted gas flow setpoint value of the first gas.

2. The control unit of claim 1, wherein the method furthermore comprises: a first step of a total flow prediction; a second step for flow scaling; and a third step for determining a predicted gas flow setpoint value and/or a scaled predicted gas flow setpoint value.

3. The control unit of claim 2, wherein in the first step, a predicted total gas flow setpoint value of a gas mixture of the at least two gases is determined starting from a set pressure value and a ventilation situation.

4. The control unit of claim 2, wherein the first step comprises a patient flow model, wherein a set pressure value and a ventilation situation are at least partially incorporated in the patient flow model and wherein a result of the patient flow model and at least partially the ventilation situation are incorporated in a calculation of a predicted total gas flow setpoint value.

5. The control unit of claim 2, wherein in the first step, starting values for a patient flow model are determined via a patient model from a ventilation situation.

6. The control unit of claim 2, wherein in the second step, a scaled predicted total gas flow setpoint value and/or the scaled predicted gas flow setpoint value is calculated from a predicted total flow setpoint value alone or from the predicted total gas flow setpoint value together with the predicted gas flow setpoint value.

7. The control unit of claim 2, wherein, in the second step a scaled predicted total gas flow setpoint value and/or a scaled predicted gas flow setpoint value is calculated from the predicted gas flow setpoint value.

8. The control unit of claim 2, wherein the second step comprises a scaling, wherein separate scaling factors and/or scaling functions are determined for inspiration and expiration and a switch is made via a switch between the scaling factors and/or scaling functions depending on inspiration or expiration.

9. The control unit of claim 8, wherein at least one comparison between a provided inspiration or expiration volume and a real applied inspiration or expiration volume is taken into consideration to determine the scaling factors and/or the scaling functions.

10. The control unit of claim 2, wherein the third step comprises a calculation of the predicted gas flow setpoint value from a predicted total flow setpoint value.

11. The control unit of claim 2, wherein the third step comprises a determination of a mean concentration mcO2% of the first gas in the second gas, wherein an exhalation volume V_Rück is also incorporated in the determination of the mean concentration mcO2% of the first gas.

12. The control unit of claim 2, wherein in the third step, a determined mean concentration mcO2% of the first gas is incorporated in a determination of the predicted gas flow setpoint value.

13. The control unit of claim 1, wherein an input variable for the feedforward component for determining the manipulated variable for the control of the gas valve is the scaled predicted gas flow setpoint value, optionally with consideration of an exhalation volume V_Rück.

14. The control unit of claim 1, wherein an input variable for the correction regulator component comprises at least one parameter, which describes a deviation of a gas flow actual value determined by at least one flow sensor from the predetermined gas flow setpoint value.

15. The control unit of claim 1, wherein the correction regulator component of the manipulated variable for the control of the gas valve becomes zero when a gas flow value corresponds to a gas flow setpoint value.

16. The control unit of claim 1, wherein the first gas is oxygen and the second gas is ambient air or compressed air/pressurized air or a gas mixture of ambient air and/or compressed air/pressurized air and/or an at least partially exhaled respiratory gas.

17. The control unit of claim 1, wherein the control unit is configured to calculate an exhalation volume V_Rück on the basis of measurement data of at least one flow sensor.

18. The control unit of claim 2, wherein
a. in the first step a predicted total flow setpoint value is calculated from at least one ventilation situation, a set pressure value, and at least partially via a patient flow model;
b. in the second step, a scaled predicted total flow setpoint value is determined from a predicted total flow setpoint value and inspiration data and expiration data via a scaling, the scaling being adapted to a respective respiration phase;
c. in the third step, the scaled predicted gas flow setpoint value is determined from a scaled predicted total flow setpoint value and a mean concentration mcO2% of the first gas determined using an exhalation volume V_Rück;
d. in a fourth step, an input variable for the feedforward component is the scaled predicted gas flow setpoint value.

19. A ventilator, wherein the ventilator comprises at least one control unit according to claim 1.

20. A method for regulating a gas flow of at least one first gas to be admixed to at least one second gas, wherein the method comprises at least one step of a control of a gas valve, wherein at least one manipulated variable for the control of the gas valve is determined from at least one correction regulator component and at least one feedforward component, an input variable of the feedforward component being a predicted gas flow setpoint value of the first gas.
